(19) United States
(12) Patent Application Publication
(10) Pub. No.: US 2025/0312181 A1
(43) Pub. Date: Oct. 9, 2025

(54) UPPER BODY CHASSIS

(71) Applicant: Mawashi science & technologie inc., Saint-Jean-sur-Richelieu (CA)

(72) Inventors: Philippe LAVOIE, Bromont (CA); Dominic FERRON, Chambly (CA); Renaud CYR, Saint-Hubert (CA); Victoria BUDICO, Montréal (CA); Christopher MANN, Saint-Jean-sur RicheLieu (CA); Alessandro TELONIO, Sherbrooke (CA); Marie-Pier LAGUE, St-Lambert (CA); Remi PAQUET, Brossard (CA); Alain OUELLET, Longueuil (CA); Juan HENAO, Montréal (CA); Patrice PAQUETTE, Melbourne (CA); Barbara LABRECQUE, Chambly (CA); Frédéric MAISONNEUVE, Laval (CA); Mylène BLANCHETTE, Chambly (CA); Alexandre TARDIF, Granby (CA); Alain BUJOLD, Dollard-des-Ormeaux (CA)

(21) Appl. No.: 18/863,310

(22) PCT Filed: May 30, 2023
(86) PCT No.: PCT/CA2023/050741

§ 371 Date: Nov. 5, 2024

Related U.S. Application Data
(60) Provisional application No. 63/347,055, filed on May 31, 2022.

(51) Int. Cl. A61F 5/02

(52) U.S. Cl. CPC A61F 5/026; A61F 5/028

(57) ABSTRACT

There is described an upper body chassis (UBC) to be worn by a user for lessening the fatigue of wearing protection equipment and delivering an unparalleled distribution of gear related loads onto the lumbo-sacral area, providing a superior comfort and support to the user. The UBC comprises a thoracic cage configured to enclose the user's thorax; a pelvic cage configured to wrapped around about the user's belt line; and a lumbar spine component coupling the pelvic cage to the thoracic cage. The lumbar spine component comprises vertebrae and intervertebral joints configured to couple two of the plurality of vertebrae. The intervertebral joints comprises a body providing a plurality of degrees of freedom for movement of the coupled vertebrae relative to each other. The UBC comprises additional components participating in lengthening of the UBC spine to compensate for the structure offset from the anatomical spine.

### CROSS-REFERENCE TO RELATED APPLICATION

This application claims priority from U.S. provisional patent application 63/347,055 filed May 31, 2022, entitled UPPER BODY CHASSIS, the specification of which is hereby incorporated herein by reference in its entirety.

### BACKGROUND

### (a) Field

The present invention generally relates to exoskeleton systems, and more particularly to passive exoskeleton of the nature of an upper body chassis.

### (b) Related Prior Art

There has been huge advancement recently in the knowledge of biological mechanisms of the human body, a.k.a. biomechanics, and, in particular, in the understanding of the role of the fascia tissue in the human body, and their interactions with the skeleton framework and the muscle system, how dynamic equilibriums are generated, the whole in mutual synergy of every part of the human body.

Soldiers must operate in particular situations wearing e.g., a ballistic plate and/or a fragmentation vest. Wearing this type of gear generates fatigue to the soldiers, which is to be lessen to the minimum.

There is therefore a need for a solution configured to support and redirect the load coming from a load bearing vest (LBV). Such need may be translated into a need for a solution configured to smartly distribute the weight of various load carriage systems and personal protective equipment (PPE) onto the spine, waist, and pelvic region of the wearer.

There is also a need for a solution that features a low profile to be worn under, e.g., the fragmentation vest.

There is therefore a need for a load-bearing solution particularly adapted for all soldiers wearing ballistic protection system, namely infantry soldiers, combat engineers, medics, and combat support soldiers.

There is further a need for other applications involving important, or time-extended load carriage, such as hiking in which a solution to decrease the shoulder fatigue and discomfort coming from the shoulder compression when transporting of a heavy backpack over long distances is sought after.

Furthermore, there is also a need for a solution that is projected to significantly improve the users' comfort and well-being, to reduce musculoskeletal injuries related to load carriage, and in turn, to increase the users' performance and well-being.

Therefore, there is a need for an improved apparatus, and particularly an upper body chassis, that provides at least some of the discussed improvements, and hence would mitigate some of the shortcomings of the prior art.

### SUMMARY

According to an embodiment, there is provided components of an upper body chassis configured to provide an aid to the user. The upper body chassis, when worn by a user, is configured to distribute load, to be fully customizable to the morphology of the user, and configured to adapt, to permit and follow movements of the user. The upper body chassis is configured to minimize pressure points over the skin of the user and to feature an air circulation level providing an improved level of comfort to the user.

According to embodiments, the components of or the upper body chassis are configured to alleviate one or more of the shortcoming encountered nowadays with available technologies.

In some aspects, the techniques described herein relate to an upper body chassis to be worn by a user, including: a thoracic cage configured to at least partially enclose the user's thorax; a pelvic cage configured to wrapped around the user's body about the user's belt line; and a lumbar spine component coupling the pelvic cage to the thoracic cage, the lumbar spine component including: a plurality of vertebrae; and at least one intervertebral joint configured to couple two of the plurality of vertebrae, the intervertebral joint including a body providing a plurality of degrees of freedom for movement of the coupled vertebrae relative to each other.

In some aspects, the techniques described herein relate to an upper body chassis, wherein the plurality of vertebra includes at least three vertebra, and the at least one intervertebral joint includes at least two intervertebral joints.

In some aspects, the techniques described herein relate to an upper body chassis, wherein the thoracic cage includes a first spine section configured to extend in a caudal-cranial direction along the user's spine, the first spine section being configured to be incompressible in the caudal-cranial direction.

In some aspects, the techniques described herein relate to an upper body chassis, wherein the thoracic cage includes a first side ribs assembly including: an upper rib and a lower rib extending laterally and toward an area anterior to the first spine section, the upper rib and the lower rib being rigidly coupled to the first spine section, and a rib coupling configured to couple the upper rib to the lower rib while providing at least one degree of freedom.

In some aspects, the techniques described herein relate to an upper body chassis, wherein the rib coupling is a pivotable coupling.

In some aspects, the techniques described herein relate to an upper body chassis, including a second side ribs assembly, wherein the first side ribs assembly and the second side ribs assembly extend over opposite sides of the user, and wherein the first side ribs assembly and the second side ribs assembly are coupled to each other at the user's front, thereby wrapping around the user's thorax.

In some aspects, the techniques described herein relate to an upper body chassis, wherein the lumbar spine includes a second spine section component configured to extend in a caudal-cranial direction along the user's spine, the second spine section being configured to be: a) at least one of i) expandable and ii) compressible in the caudal-cranial direction; and b) rotatable around a connection line connecting one of the vertebrae to one of the intervertebral joint, the connection line extending in the caudal-cranial direction.

In some aspects, the techniques described herein relate to an upper body chassis, wherein the second spine section includes a sacrum component configured to butts up against the user's sacrum, the sacrum component being able to adapt to position and angle of the user's sacrum as the user moves.

In some aspects, the techniques described herein relate to an upper body chassis, wherein the intervertebral joint is configured to prevent the lumbar spine component to bend toward a posterior area while allowing the lumbar spine component to bend toward an anterior area through caudal-cranial move of the vertebrae away from each other.

In some aspects, the techniques described herein relate to an upper body chassis, wherein the pelvic cage includes a sacrum component coupled to the lumbar spine component, the sacrum component being configured to butts up against the user's sacrum and to be able to adapt to position and angle of the user's sacrum as the user moves.

In some aspects, the techniques described herein relate to an upper body chassis, wherein the pelvic cage includes a waist wrapping component, coupled to the sacrum component, configured to wrap around the user's waist thereby maintain the sacrum component abutting the user's sacrum.

In some aspects, the techniques described herein relate to an upper body chassis, wherein the waist wrapping component including two belt extremities, and a closure component configured to releasably couple and maintain coupling of the two belt extremities.

In some aspects, the techniques described herein relate to an upper body chassis, wherein the waist wrapping component includes a 3D shape assembly of strands.

In some aspects, the techniques described herein relate to an upper body chassis, wherein the 3D shape assembly of strands is configured for the waist belt to interface with a plurality of: a depression of the user's sacral region; protrusions of the user's iliac crests; the user's anterior inferior iliac spines (ASIS); and a depression under the user's abdomen.

In some aspects, the techniques described herein relate to an upper body chassis, wherein the sacrum component includes: a sacrum lower body configured to interface with the user's sacrum; a sacrum upper body rotatably coupled to the sacrum lower body; and a coupling component mounted to the sacrum upper body, configured to receive a load from the lumbar spine component.

In some aspects, the techniques described herein relate to an upper body chassis, wherein the sacrum component includes a sacrum body and a pair of wings extending laterally from the sacrum body, the wings being coupled pivotably to the sacrum body, wherein the wings are configured to interface with the user's back.

In some aspects, the techniques described herein relate to an upper body chassis, wherein the sacrum body includes a sacrum lower body and a sacrum upper body coupled rotatably to each other; wherein rotatable coupling of the sacrum upper body and sacrum lower body defines a first axis; wherein rotatable coupling of the wings to the sacrum body defines a second axis; and wherein the first axis and the second axis are parallel.

In some aspects, the techniques described herein relate to an upper body chassis, wherein the intervertebral joint includes: a first extremity and a second extremity; and a body connecting the first extremity to the second extremity, the body providing a plurality of degrees of freedom to the intervertebral joint for movement of the extremities relative to each other.

In some aspects, the techniques described herein relate to an upper body chassis, wherein the vertebrae includes a first element and a second clement configured to be assembled together to couple the vertebrae to the intervertebral joint.

In some aspects, the techniques described herein relate to an upper body chassis, wherein the plurality of vertebrae are configured to adjust so that when a load is donned onto the upper body chassis, the lumbar spine component leans slightly towards the user's spine.

In some aspects, the techniques described herein relate to an upper body chassis, wherein the thoracic cage includes a thoracic spine component, a shoulder bridge components, a left-side ribs assembly, a right-side ribs assembly, and a sternum component; wherein the shoulder bridge components are connected to a first portion of the thoracic spine component, and are configured to wrap the use's thorax above the user's shoulders; wherein the left-side ribs assembly and the right-side ribs assembly are connected to a second portion of the thoracic spine component that is lower than the first portion, the left-side ribs assembly and the right-side ribs assembly being configured to wrap around the user's thorax about the user's ribs; and wherein the shoulder bridge components, the left-side ribs assembly, the right-side ribs assembly couple to the sternum component about the user's sternum.

In some aspects, the techniques described herein relate to an upper body chassis to be worn by a user, including: a spine component configured to extend in a direction along the user's spine, the spine component including a first spine section configured to be not compressible in the caudal-cranial direction, and a second spine section configured to be (expandable) compressible in the caudal-cranial direction and rotatable around the caudal-cranial direction, a thoracic cage configured to enclose the thorax of the user, the thorax cage including the first section of the spine component, wherein the second spine section includes a sacrum component configured to butts up against the user's sacrum and to be able to adapt to position and angle of the sacrum of the user as the user moves, whereby the sacrum component is configured to replicate natural kinematics of the user's sacroiliac joint (SIJ) under vertical loading and to allow the upper body chassis to transfer loads to an anterior aspect of the user's sacrum to support SIJ function.

In some aspects, the techniques described herein relate to an upper body chassis to be worn by a user, including: a spine component configured to extend in a caudal-cranial direction along the user's spine, the spine component including a first spine section including several vertebrae coupled through intervertebral joints, and a second spine section, a thoracic cage configured to enclose the thorax of the user, the thorax cage including the first section of the spine component, and two ribs assembly coupled to each other anterior to the user, each one of the ribs assembly including at least two ribs rotatably coupled to each other, and wherein the at least two ribs are coupled to the vertebrae of first section of the spine, and wherein the intervertebral joint is configured to prevent bent of the spine component toward posterior and to allow bent of the spine component toward anterior through caudal-cranial move of the vertebrae away from each other.

In some aspects, the techniques described herein relate to a waist belt configured to interface with a load-bearing component, the waist belt including: a coupling component configured to interface with the load-bearing component; a sacrum component, coupled to the coupling component, configured to butts up against the user's sacrum and to be able to adapt to position and angle of the sacrum of the user as the user moves; and a waist wrapping component, coupled to the sacrum component, configured to wrapped up the user's waist thereby maintain the sacrum component abutting the user's sacrum.

In some aspects, the techniques described herein relate to a sacrum component of a load-bearing aid configured to be worn by a user when bearing a load, the sacrum component including: a sacrum lower body configured to interface with the sacrum of the user; a sacrum upper body rotatably coupled with the sacrum upper body; a coupling component mounted to the sacrum upper body, configured to receive the load; and a pair of wings, extending laterally from the sacrum lower body, coupled pivotably to the sacrum lower body, the wings being configured to interface with the back of the user.

In some aspects, the techniques described herein relate to a (lumbar) spine component of a load-bearing aid configured to be worn by a user when bearing a load, the lumbar spine component including: a pair of intervertebral joints each including a first extremity and a second extremity; and a body connecting the extremities, the body providing a plurality of degrees of freedom to the intervertebral joint(s) for movement of the extremities relative to each other; an vertebrae including a first element and a second clement configured to be assembled together to couple to the first extremity of the interval joints with the vertebra, and a pair of wings extending laterally from the first element, the wings being configured to provide an interface with the user's back laterally from the user's spine.

In some aspects, the techniques described herein relate to a waist belt including: a waist wrapping component configured to wrap up the user's waist, the waist wrapping component including two extremities; closure component configured to releasably couple and maintain coupling of the two extremities; wherein the waist wrapping component consists in a 3D shape assembly of strands.

In some aspects, the techniques described herein relate to a rib assembly of a load-bearing aid configured to be worn by a user when bearing a load, including: a first rib extending laterally and frontally from a dorsal central component of the load-bearing aid at a first coupling position; a second rib extending laterally and frontally from the dorsal central component of the load-bearing aid distal at a second coupling position distant from the first coupling position; a rib coupling component coupling the first rib with the second rib, the rib coupling component being distant from the first coupling position and from the second coupling position, wherein the rib coupling component maintains coherence of the rib assembly upon movement of the first coupling position and the second coupling position relative to each other.

In some aspects, the techniques described herein relate to an upper body chassis to be worn by a user, the upper body chassis including: a lumbar spine component featuring 6 degrees of freedom, the lumbar spine component including lumbar vertebrae configured to be in contact with (follow) the user's natural lumbar curve; and a mobile sacrum component coupled to the lumbar spine component; wherein the lumbar vertebrae are configured to adjust so that when a load is donned onto the upper body chassis, the lumbar spine component, in conjunction with the mobile sacrum component, lean slightly towards the user's spine, whereby a bracing effect is provided to the user's lumbar region that is maintained throughout a task such as walking as the lumbar spine component follows movements of the user's spine.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1A to 1E are respectively a front view, a front-oblique view, a left side view, a rear-oblique view and a rear view of the upper body chassis on a user with identification of reference directions;

FIG. 2A is a perspective front-oblique view of an upper body chassis worn by a user in accordance with an embodiment;

FIG. 2B is a perspective back-oblique view of an upper body chassis of FIG. 1 worn by a user;

FIG. 3 is a rear view of the upper body chassis of FIG. 1 without the waist belt;

FIG. 4 is a back-oblique view of the partial upper body chassis of FIG. 3;

FIG. 5 is a right-side view of the partial upper body chassis of FIG. 3;

FIG. 6 is a front-oblique view of the partial upper body chassis of FIG. 3;

FIG. 7 is a front elevation view of the partial upper body chassis of FIG. 3;

FIG. 8 is a partially upward, front and left-oblique perspective view of the partial upper body chassis of FIG. 3;

FIG. 9 is a front oblique view of the thoracic cage of the upper portion of the upper body chassis of FIG. 3;

FIG. 10 is a rear oblique view of the thoracic cage of the upper portion of the upper body chassis of FIG. 3;

FIG. 11 is a rear view of a user wearing a waist belt of an upper body chassis in accordance with an embodiment;

FIG. 12 is a left elevation side view depicting a portion of an upper body chassis 100 worn by a user, comprising a waist belt and a sacrum component;

FIG. 13 is a rear-left oblique view depicting a portion of an upper body chassis worn by a user, comprising a waist belt and a sacrum component;

FIG. 14 is a perspective view depicting a portion of an upper body chassis showing the inner face of the housing and a portion of the lumbar spine component in accordance with an embodiment;

FIG. 15 is a schematic of the configuration of the flat pattern strands of the waist belt ilia of a waist belt in accordance with an embodiment;

FIGS. 16-19 are rear oblique respectively a perspective view, a rear view, a front view, and a side view of a waist bell ilia wrapped around an invisible waist in accordance with an embodiment;

FIG. 20 is a perspective view of the sacrum component of the upper body chassis of FIG. 1;

FIG. 21 is a perspective view of a wing of the sacrum component of FIG. 20;

FIG. 22 is a perspective view of the central assembly of the sacrum component of FIG. 20;

FIG. 23 is an elevate view of the central assembly of FIG. 22;

FIG. 24 is a side view of the central assembly of FIG. 22;

FIG. 25 is a cross-section perspective view along cross-section line A-A of the central assembly of FIG. 23;

FIG. 26 is a cross-section side view along cross-section line A-A of the central assembly of FIG. 23;

FIG. 27 is a front oblique view of the lumbar spine component of the upper body chassis of FIG. 1 coupled with the sacrum component and the posterior ribs component;

FIG. 28 is a front oblique view of the lumbar spine component of the upper body chassis of FIG. 1;

FIG. 29 is a front oblique view of a combination of two neighbor vertebrae coupled to a lumbar intervertebral joint of the lumbar spine component of FIG. 28;

FIG. 30 is a front oblique view of the combination of two neighbor vertebrae coupled and a lumbar intervertebral joint of the lumbar spine component of FIG. 28 with the internal cap removed;

FIG. 31 is a perspective view of a lumbar intervertebral joint of the lumbar spine component of FIG. 28;

FIG. 32 is a rear oblique view of the lumbar intervertebral joint of FIG. 31;

FIG. 33 is a side view of the lumbar intervertebral joint of FIG. 31;

FIG. 34 is a perspective view of an external cap of a vertebrae of the lumbar spine component of FIG. 28 without pads mounted thereto;

FIGS. 35 and 36 are respectively a rear perspective view and a front perspective view of an internal cap of the lumbar spine component of FIG. 28;

FIG. 37 is a rear oblique view of a portion of an upper body chassis of FIG. 1 worn by a user, depicting the interface of the upper body chassis with the back of the user;

FIG. 38 is a front oblique view of a portion of an upper body chassis of FIG. 1 depicting the interface of the upper body chassis with the back of an invisible user when worn;

FIGS. 39A and 39B are respectively a front oblique perspective view and a rear oblique perspective view of the posterior ribs component of the upper body chassis of FIG. 1 in normal configuration;

FIG. 40 is a front oblique perspective view of the posterior ribs component of the upper body chassis of FIG. 1 when the upper body chassis is bent in the anterior direction;

FIG. 41 is a rear oblique perspective view of the left rib assembly comprising the left upper rib and left lower rib of the posterior ribs component of FIGS. 39A-B and 40A-B;

FIG. 42 is a front exploded perspective view of a rib vertebrae and of an intervertebral joint of the posterior ribs component of FIGS. 39A-B and 40A-B;

FIGS. 43 and 44 are respectively a rear oblique perspective view and a front oblique perspective view of the thoracic spine component of the of the upper body chassis of FIG. 1;

FIGS. 45 and 46 are respectively a front oblique perspective view and a rear oblique perspective view of the upper vertebrae of the thoracic spine of FIGS. 43 and 44;

FIG. 47 is an upward rear oblique perspective view of the upper vertebrae of the thoracic spine of FIGS. 43 and 44;

FIG. 48 is a perspective view of lower vertebrae of the thoracic spine of FIGS. 43 and 44;

FIG. 49 is a perspective view of the shoulder bridge component of the of the upper body chassis of FIG. 1;

FIG. 50 is a perspective view of one part of the shoulder bridge component of FIG. 49;

FIGS. 51 and 52 are respectively a front oblique perspective view and a rear oblique perspective view of an assembly of the anterior ribs component and the sternum component of the upper body chassis of FIG. 1;

FIG. 53 is a front view of the assembly of the anterior ribs component and the sternum component of FIGS. 51 and 52;

FIG. 54 is a rear oblique perspective view of the anterior ribs component of the assembly of FIGS. 51 and 52;

FIGS. 55 and 56 are respectively a front oblique perspective view and a rear oblique perspective view of the sternum component of FIGS. 51 and 52;

FIGS. 57A-G depicts steps performed by a user when donning the upper body chassis of FIG. 1; and

FIGS. 58A-D depicts steps performed by a user when doffing the upper body chassis of FIG. 1.

It will be noted that throughout the appended drawings, like features are identified by like reference numerals.

### DETAILED DESCRIPTION

The realizations will now be described more fully hereinafter with reference to the accompanying figures, in which realizations are illustrated. The foregoing may, however, be embodied in many different forms and should not be construed as limited to the illustrated realizations set forth herein.

With respect to the present description, references to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values and of values herein or on the drawings are not intended to be limiting, referring instead individually to any and all values falling within the range, unless otherwise indicated herein, and each separate value within such a range is incorporated into the specification as if it were individually recited herein. The words “about”, “approximately”, or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described realizations. The use of any and all examples, or exemplary language (“e.g.,” “such as”, or the like) provided herein, is intended merely to better illuminate the exemplary realizations and does not pose a limitation on the scope of the realizations. The use of the term “substantially” is intended to mean “for the most part”. It is to be construed as indicating that some deviation from the word it qualifies is acceptable as would be appreciated by one of ordinary skill in the art to operate satisfactorily for the intended purpose.

In the following description, it is understood that terms such as “first”, “second”, “top”, “bottom”, “above”, “below”, and the like, are words of convenience and are not to be construed as limiting terms.

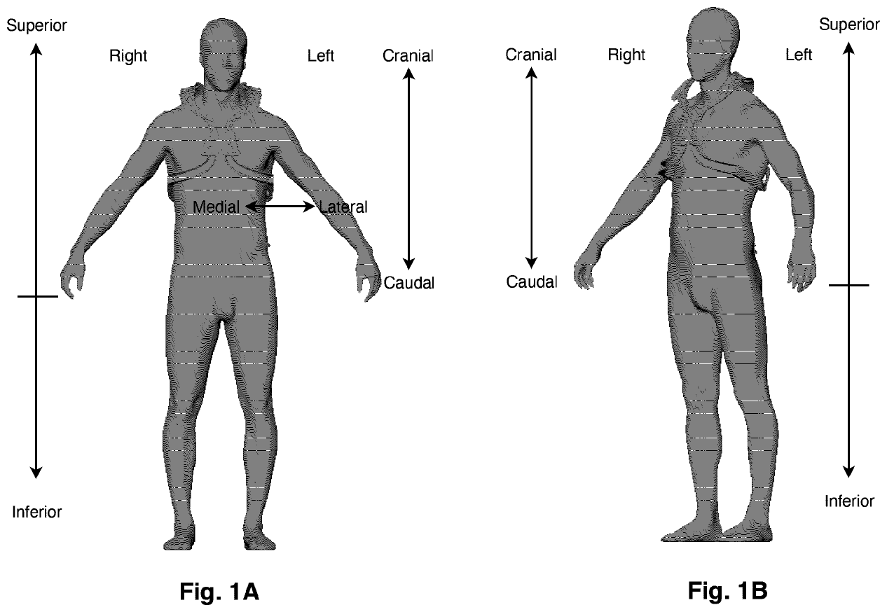

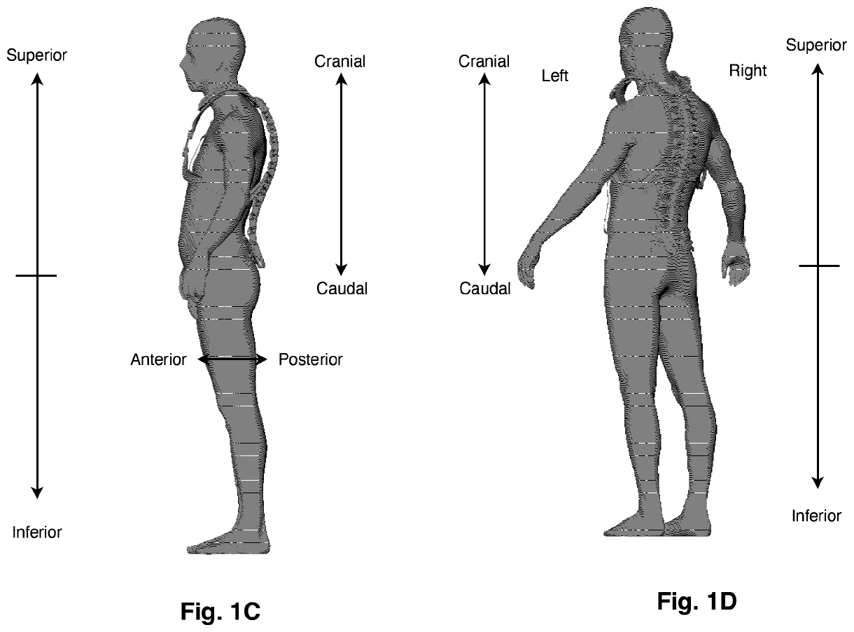

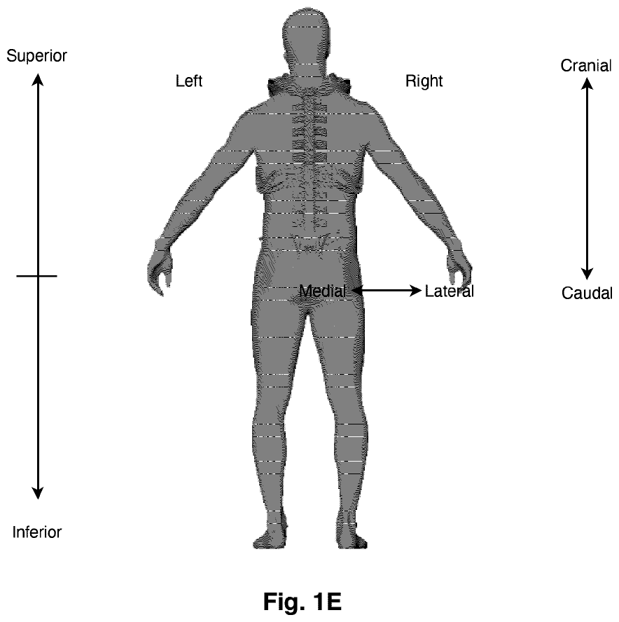

The terms “superior”, “inferior”, “upper”, “lower”, “down”, “vertical”, “horizontal”, “right”, “left”, “caudal”, “cranial”, “medial”, “lateral”, “anterior” and “posterior” and the like are intended to be construed in their normal meaning in relation with normal wearing of the product as depicted on FIGS. 1A-E.

It should further be noted that for purposes of this disclosure, the term “coupled” means the joining of two members directly or indirectly to one another. Such joining may be stationary in nature or movable in nature unless specified. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature unless specified.

In realizations, there are disclosed upper body chassis 100 to be worn by a user.

The upper body chassis 100 comprises a series of subcomponents coupled to each other, forming together an upper body chassis 100 adaptable and customizable to the features and morphologies of different users.

The upper body chassis 100 is configured to adapt to the unique shape of every user to closely mimic the biomechanics of the user's spine and adjacent structures, namely ribcage and pelvis.

It is worth noting that the upper body chassis 100 is “self-supporting”, or in other words, the upper body chassis 100 does not require a harness to interface with the user.

Referring now to the drawings, and more particularly to FIGS. 1A-E, and 2A-B to 10, the upper body chassis 100 comprises a waist belt 110, a sacrum component 120, a lumbar spine component 190, posterior ribs component 140, a thoracic spine component 210, a shoulder bridge component 160, an anterior ribs component 170, and a sternum component 180.

The posterior ribs component 140, thoracic spine component 210, shoulder bridge component 160, anterior ribs component 170 and sternum component 180 are coupled together to define a thoracic cage 105 wrapping the user front and back. The waist belt 110 and the sacrum component 120 are coupled together to define a pelvic cage 103 wrapping up the user front and back, about the belt line lower to the thoracic cage 105. The lumbar spine component 190 couples the pelvic cage 103 to the thoracic cage 105 to define the upper body chassis 100. The upper body chassis 100, excluding the waist belt 110, forms the upper portion 101 of the upper body chassis 100.

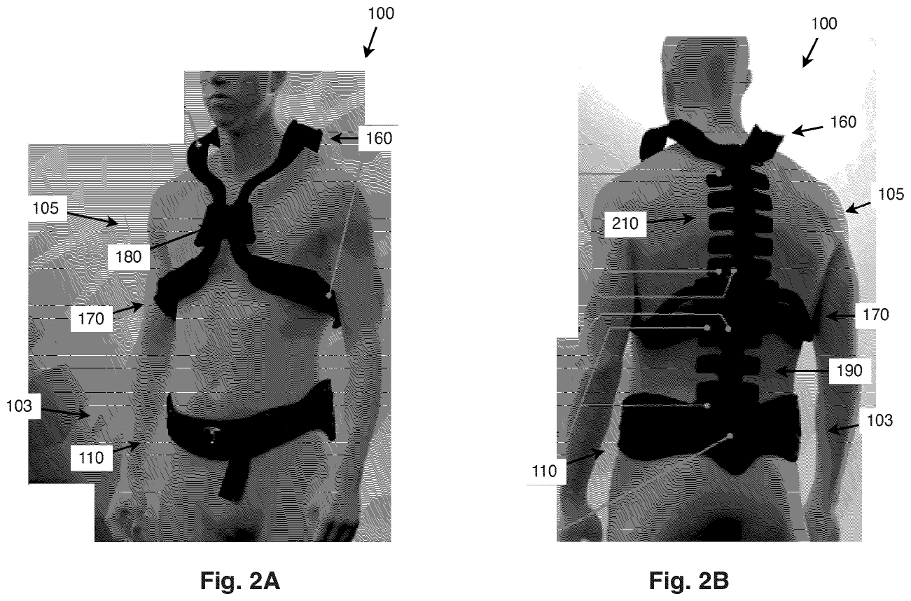

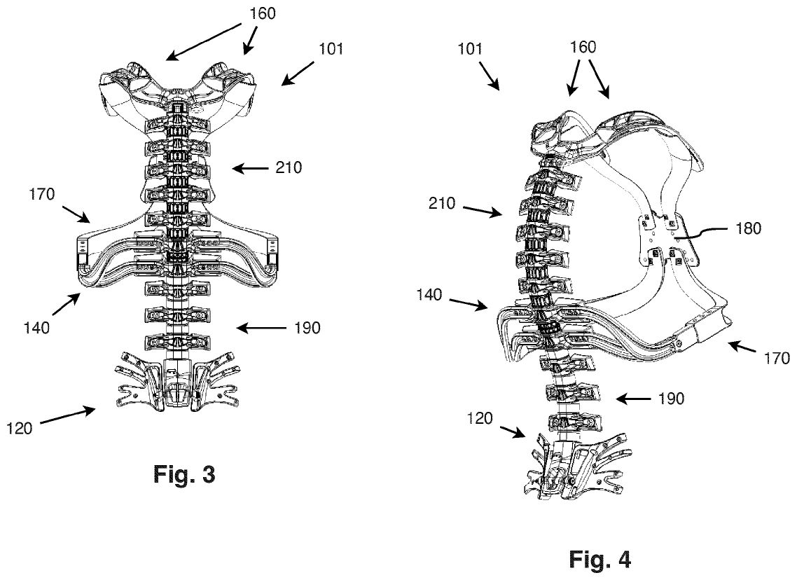

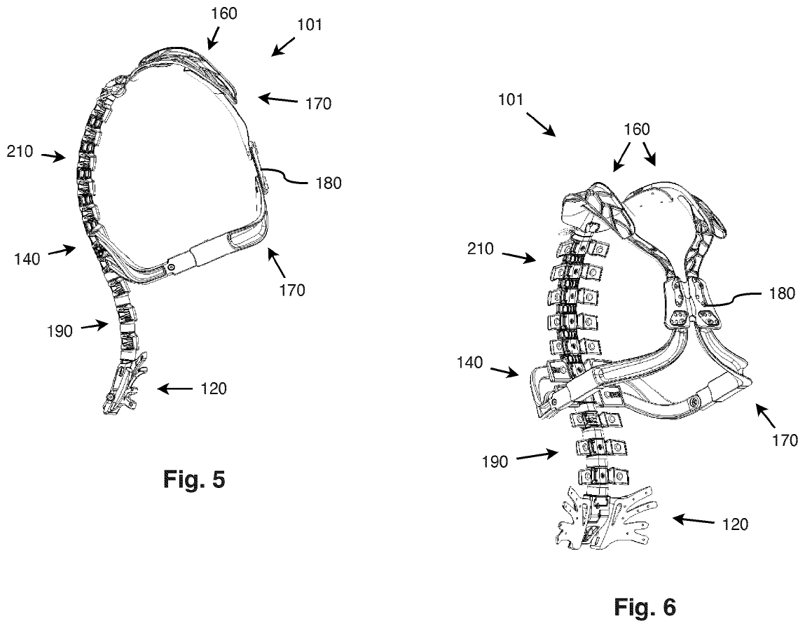

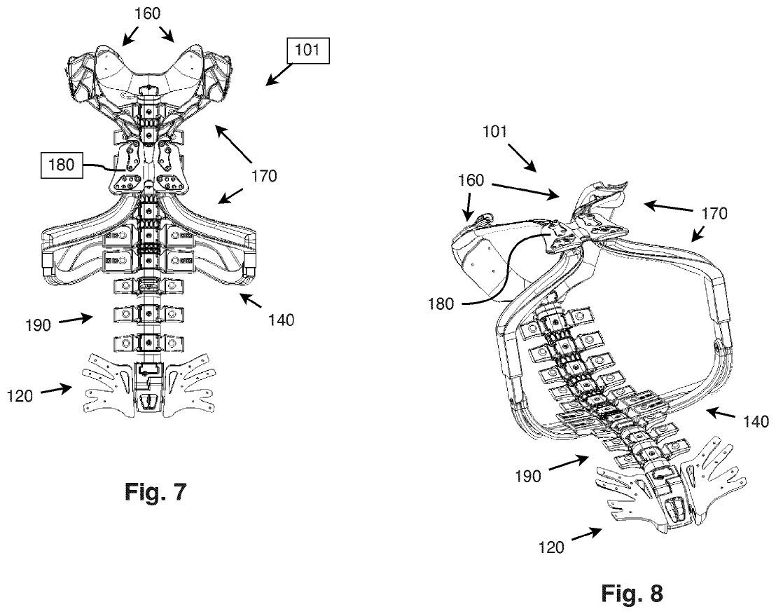

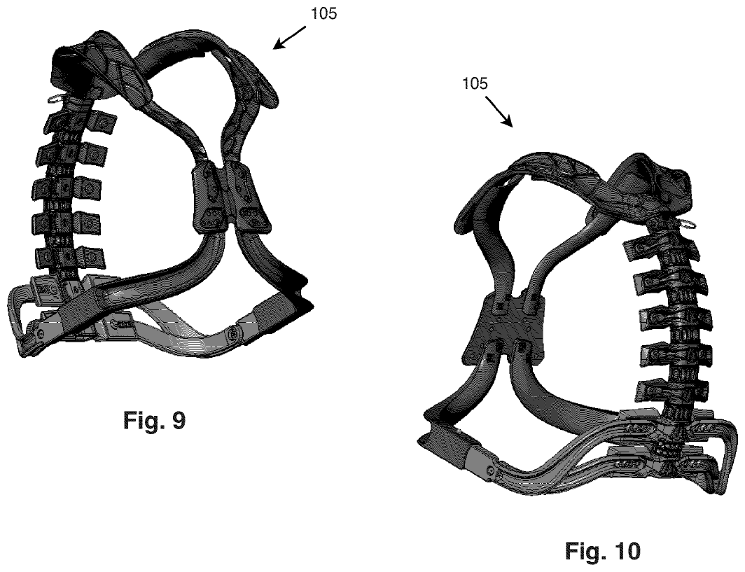

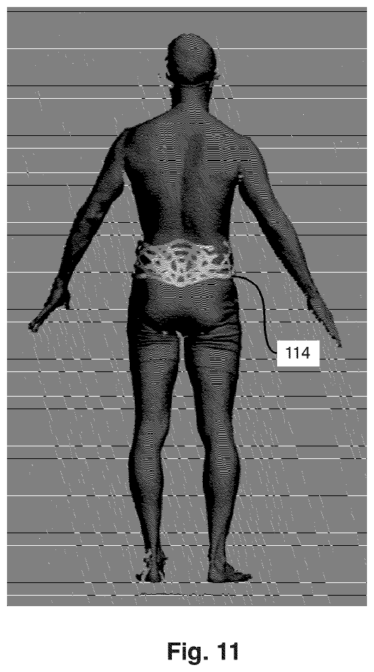

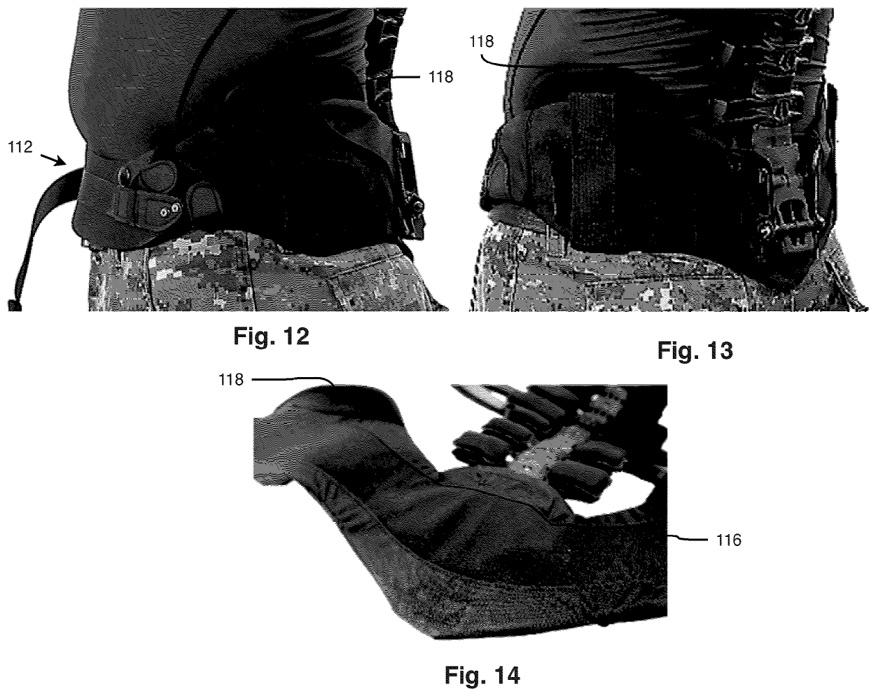

Referring to FIGS. 2A-B and 11-19, the pelvic cage 103 comprises a waist belt 110 that provides a foundation to the upper body chassis 100, as it provides a firm interface with the geometry of the user's pelvic region. The waist belt 110 interfaces with the depression of the sacral region, the protrusions of iliac crests, the anterior inferior iliac spines (ASIS) and the depression under the abdomen. While providing a firm lock, the waist belt 110 is configured to be sufficiently supple to adhere to slight morphological variations and to spread loads throughout extensive waist belt interface regions, therethrough avoiding localized and severe contact points.

The waist belt 110 comprises a front closure 112. The waist belt 110 comprises a waist belt ilia 114 consisting in a 3D shape assembly, composed of thin lightweight flexible materials such as polyamides, e.g., Nylon, manufactured as flat pattern strands, and assembled by rivets that precisely mimics the pelvic geometry. The waist belt 110 comprises a waist belt housing 116 that consists in a removable washable housing provides additional dampening by means of a pre-tensioned breathable mesh lining and of closed cell pads 118 located at the interface with the user's ASIS landmarks.

Referring to FIGS. 2A-B and 20-26, the pelvic cage 103 comprises the sacrum component 120 that consists in an adjustable component (in conjunction with the lumbar spine component 190) that replicates the natural kinematics of the sacroiliac joint (SIJ) under vertical loading and allows the upper body chassis 100 to transfer loads to the anterior aspect of the human sacrum to support SIJ function. Since the SIJ acts as a self-compensating force-couple to balance forces of gravity and weight-loading, such distribution of the loads is advantageous.

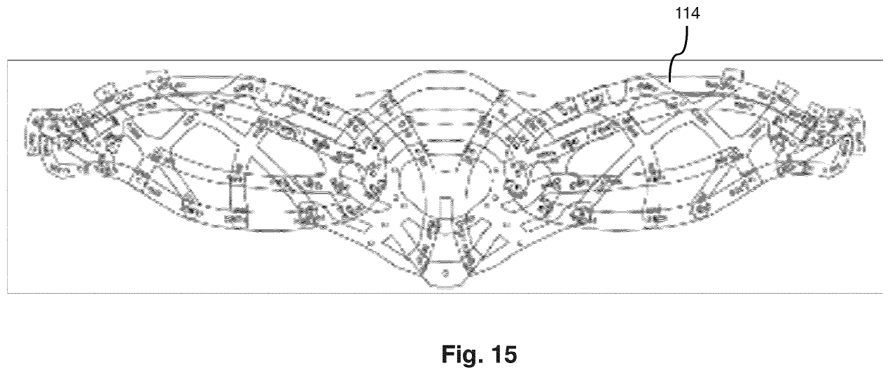

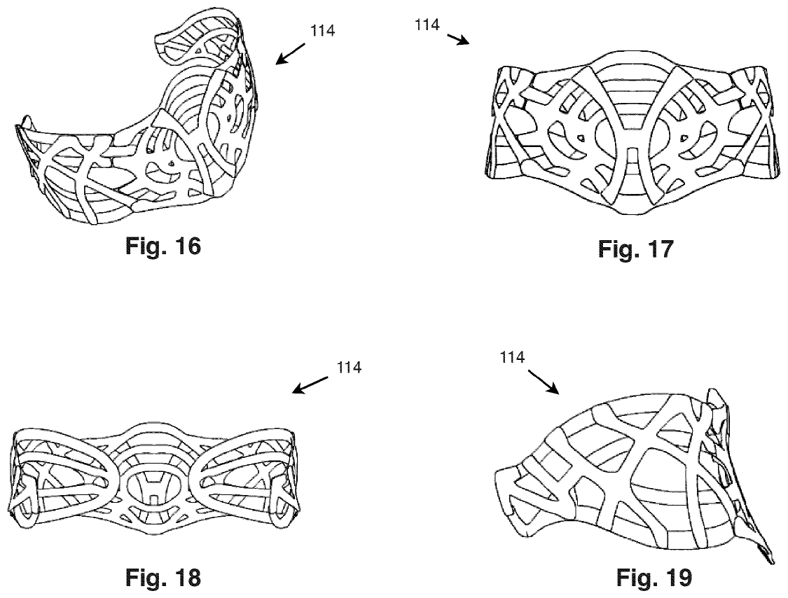

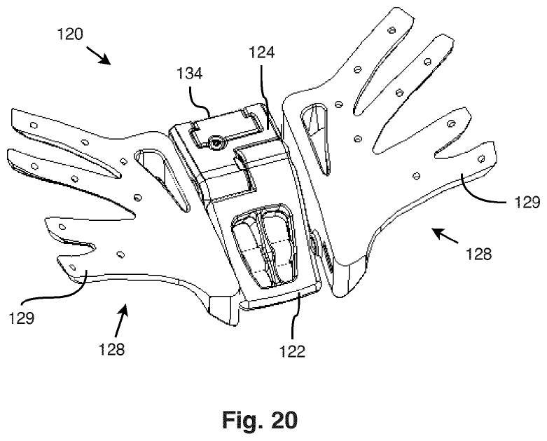

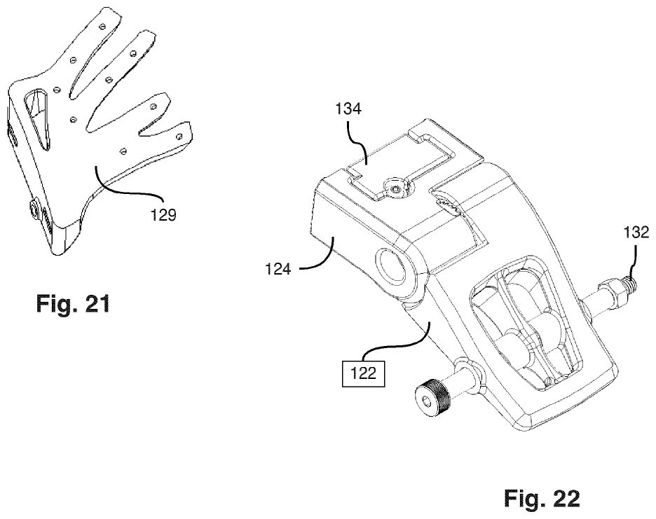

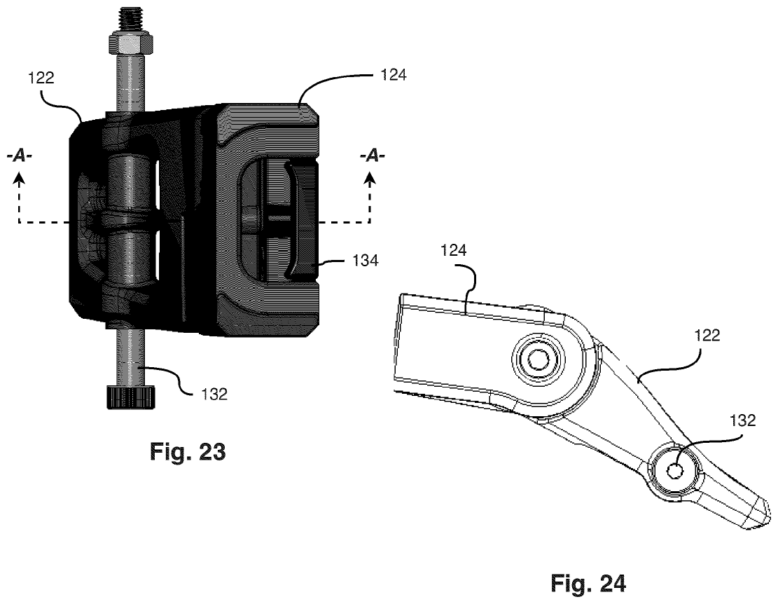

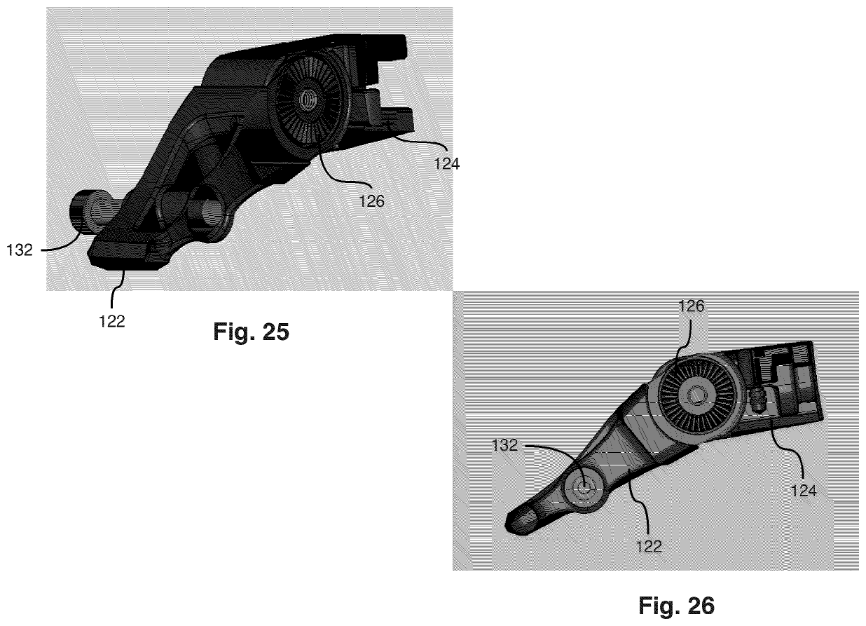

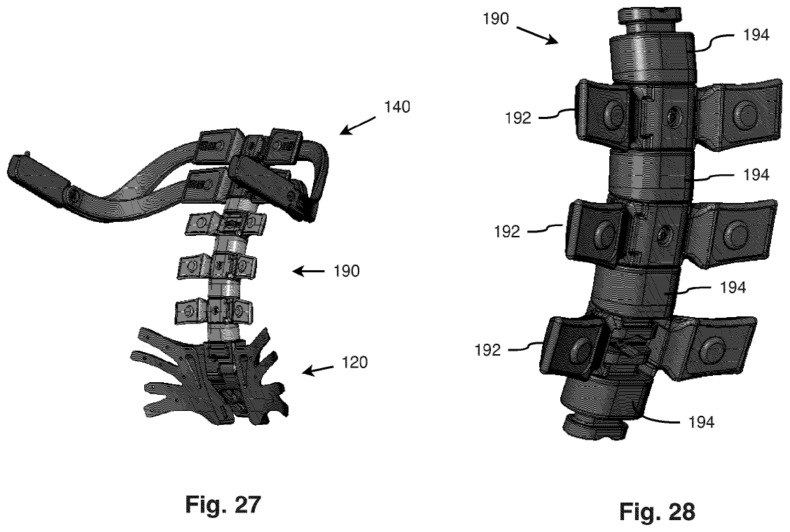

The sacrum component 120 comprises a sacrum lower body 122 consisting in a rigid polymeric part with a cavity that receives the Sacrum Pivot pin of the user. The sacrum component 120 comprises sacrum upper body 124 that consists in a rigid polymeric part with an interface to the lowest Lumbar Intervertebral Joint of the user. The sacrum component 120 comprises a lumbosacral angle adjustor 126 that consists in a lockable adjustment pivot that allows modulation of the Lumbosacral Angle to marry the shape and movement of the sacrum of the user. The sacrum component 120 also comprises a sacrum hub 128 that consists in two stiff polymeric parts (wings 129) that reflect the local geometry of the waist belt ilia 114 (see e.g., FIG. 15) to provide a solid support for the sacrum pivot 132. The sacrum component 120 also comprises the sacrum pivot 132 that consists in a shoulder screw that joins the lower body 122 to the sacrum hub 128 and that represents the natural SIJ axis.

It is worth noting that a mechanical play is incorporated in the sacrum pivot 132 to cinematically replicate the variability of the oblique force dependent SIJ axis during static load bearing and walking activities. Furthermore, the suppleness of the waist belt ilia 114 supports this SIJ axis variability.

It is worth noting that when walking the ground reaction forces coming from the foot contact on the ground are transmitted to the acetabulum of the user. This cause a rotation of the iliac crest and the counterrotation of the sacroiliac joint. In view of this situation, according to an embodiment, the axis of the rotation of the sacrum is oblique According to an embodiment, the sacrum component 120 is configured to adapt and marry the movements of the user's sacrum, comprising additionally lateral displacement of the top and bottom of the sacrum component 120 and vertical displacement of the sides of the sacrum component 120 around an oblique axis. Thus, to account of the opposite rotation of the innominate bones during the walking or during asymmetric posture, the capability of rotation of the sacro-iliac joint is multiaxial to be able to adapt to different postures and to maintain the effectiveness of the force transfer from the ground to the spine and from the spine to the legs.

Finally, the sacrum component 120 comprises at its top a jaw 134 configured for rigidly coupling the waist belt 110 with the lumbar spine component 190.

Referring to FIGS. 2A-B and 27-38, the lumbar spine component 190 consists in a modular stack-up of vertebrae 192 and of lumbar intervertebral joints 194 that allow close fitting of the user's natural lordosis and that replicates the kinematics of the natural lumbar spine. This lumbar spine component 190, inspired by the human anatomy, provides good lumbar support that braces the user's lower back when loads are added onto upper body chassis 100. The lumbar spine component 190 are provided in different angulations to adapt to different morphologies. Furthermore, the modular stack-up configuration of the lumbar spine component 190, allowing variability in the number of vertebrae 192, the angle/orientation of the lumbar intervertebral joints 194, and the stiffness of lumbar intervertebral joints 194, allows to conform to the variability of lordosis curves of users, namely different lordosis angles and curve lengths.

The lumbar spine component 190 comprises the lumbar intervertebral joints 194 that are elements that connect the vertebrae 192 together and that allows for six (6) degrees of freedom, namely expansion/compression in all three (3) axes, and rotation in all three (3) axes within a predetermined range of acceptable displacement. mimicking the kinematics of spinal functional units. Each lumbar intervertebral joints 194 is composed of a gel-like component (ex: Polyurethane) with an interface mechanism to be mechanically connected to vertebrae 192. The lumbar intervertebral joints 194 can also be made of other materials, such as elastics, semi-rigid cables, and knit assemblies.

The lumbar spine component 190 comprises vertebrae 192 that are rigid assemblies composed of an internal cap 196 and an external cap 198 that encapsulate the extremities 200 of two neighbor lumbar intervertebral joints 194.

The user interfacing aspect of each vertebrae 192 is configured for the internal cap 196 to provide a central recess 208 and the external cap 198 to comprises two (2) wings 202 extending laterally and toward the anterior that are equipped with paddings 204 configured to provide a comfortable interface with the user's back spread over an extended vertical length, aka in a cranial-caudal direction, with the contact being offset, aka lateral, from sensitive vertebral spine and dampen the contact applied to the sides of the human spine.

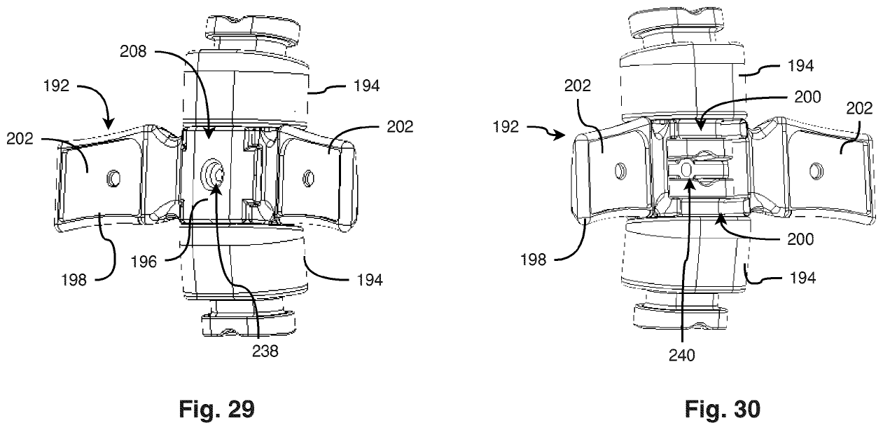

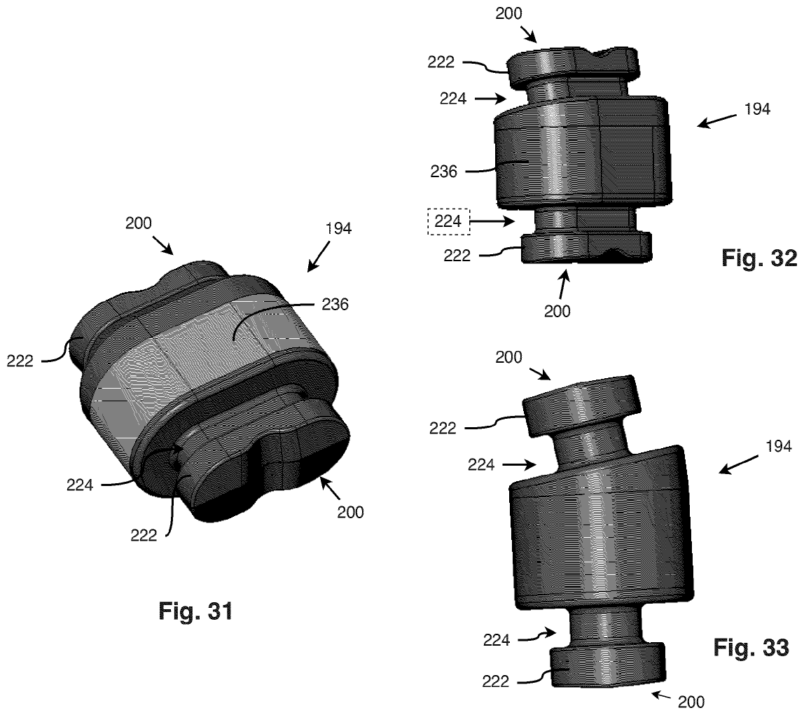

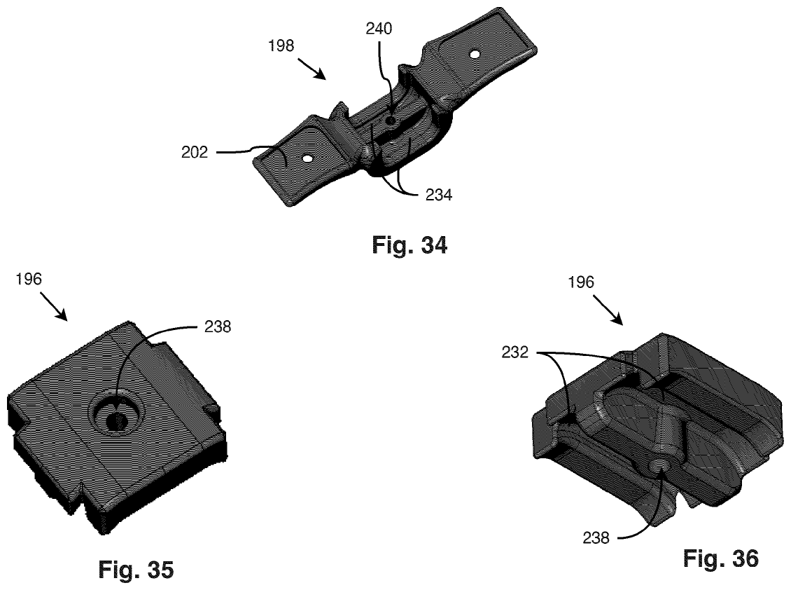

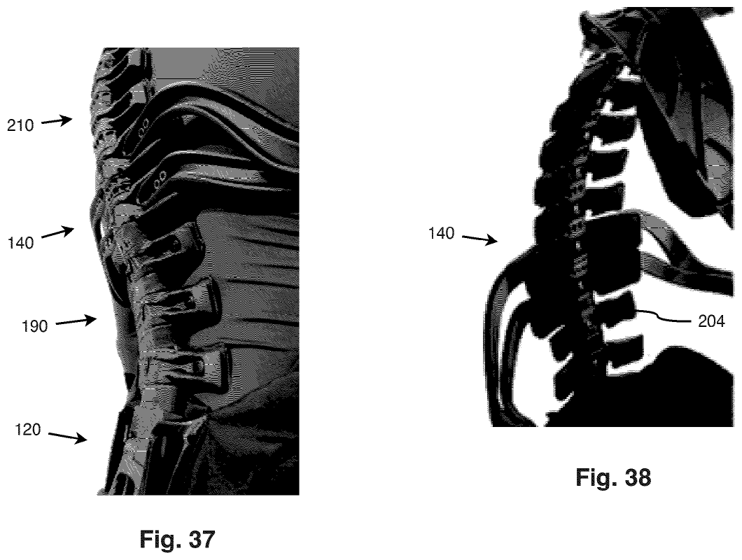

Referring particularly to FIGS. 29 to 36, here is described an example of the coupling of a vertebrae (e.g., vertebrae 192) with an intervertebral joint (e.g., lumbar intervertebral joints 194) that is replicated substantially throughout the extend of the spinal extent of the upper body chassis 100. The coupling involves the extremities 200 of the intervertebral joint 194 featuring a surrounding lip 222 defining a recess 224 beside it, wherein the lip 222 is configured to interface with channels 232, 234 present in the internal cap 196 and the external cap 198 operating as a pair of jaws. The assembly of the internal cap 196 with the external cap 198, with the extremity 200 of an intervertebral joint 194 inserted into the channels 232, 234, results in setting in the extremity 200 in the caps 196, 198. Once a screw (not shown) mounted to couple the caps 196, 198 through the mounting holes 238, 240, the lumbar intervertebral joint 194 becomes set such that it cannot be removed without uncoupling the caps 196, 198 from each other.

According to embodiments and locations, the lumbar intervertebral joints may be rigid pieces offering no degree of freedom (e.g., intervertebral joint 148. FIG. 42), or pieces wherein the body 236 may be e.g., made of a gel-like component. or a mix of a gel-like component and of rigid parts, of pivoting parts, or a combination thereof, that may offer a different number of degrees of freedom, different orientations of these degrees of freedom, and different limitations to these degrees of freedom.

Similarly, according to embodiments (mostly not depicted), the coupling of an intervertebral joint with a vertebra may be rigid (featuring no space or capacity for relative displacement between vertebrae), or may feature difference in size and/or configuration between the lip 222 and the channels 232, 234 allowing relative displacement. According to embodiments, the use of a damping material such as a gel in the channels may be used to limit the relative displacement and to limit the wear resulting from the relative displacement of the components in play.

Referring to FIGS. 2A-B and 39A-B to 42, the posterior ribs component 140 is the base of the thoracic cage 105. The posterior ribs component 140 has three (3) main functions, that are: 1—allowing a “lengthening” of the spine portion of the upper body chassis 100 to compensate for the structure offset from the anatomical spine of the user: 2—supporting the stiffness of the thoracic spine component 210, the shoulder bridge component 160 and the anterior ribs component 170; and 3—providing a stable interface with a fragmentation vest (or another equipment) worn by the user, which prevents the rigid fragmentation plate from tilting from side to side.

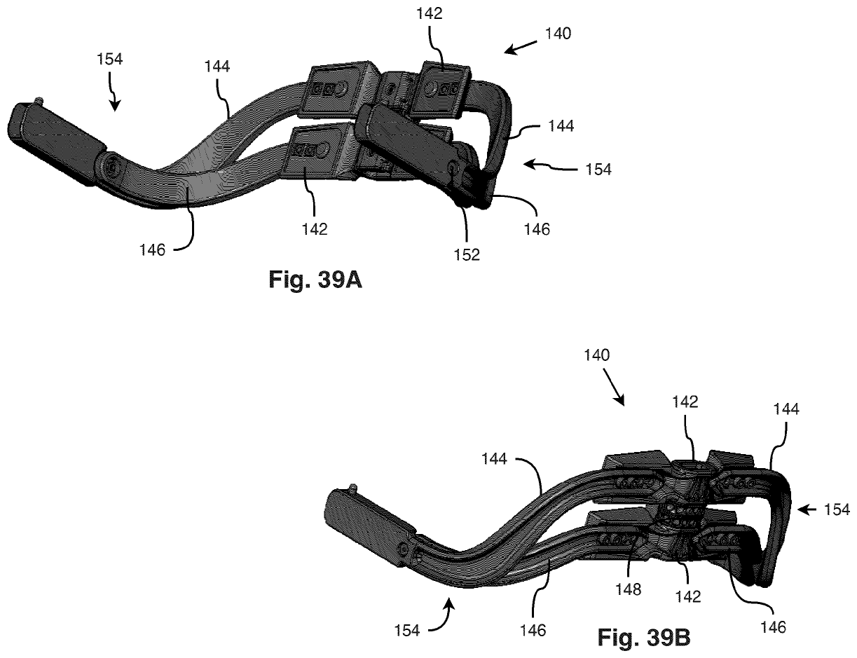

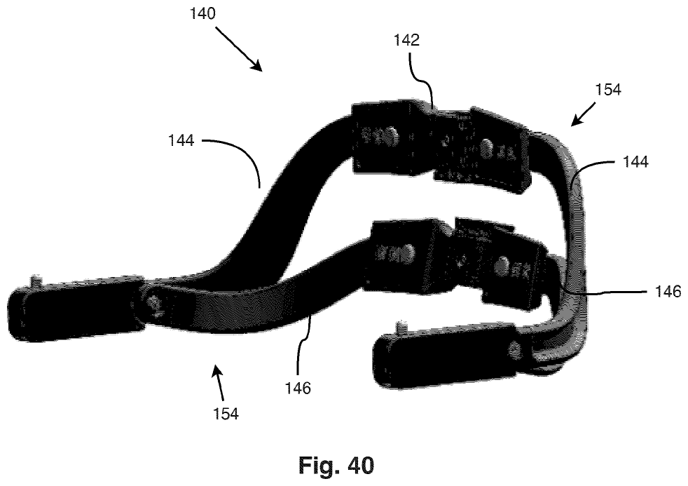

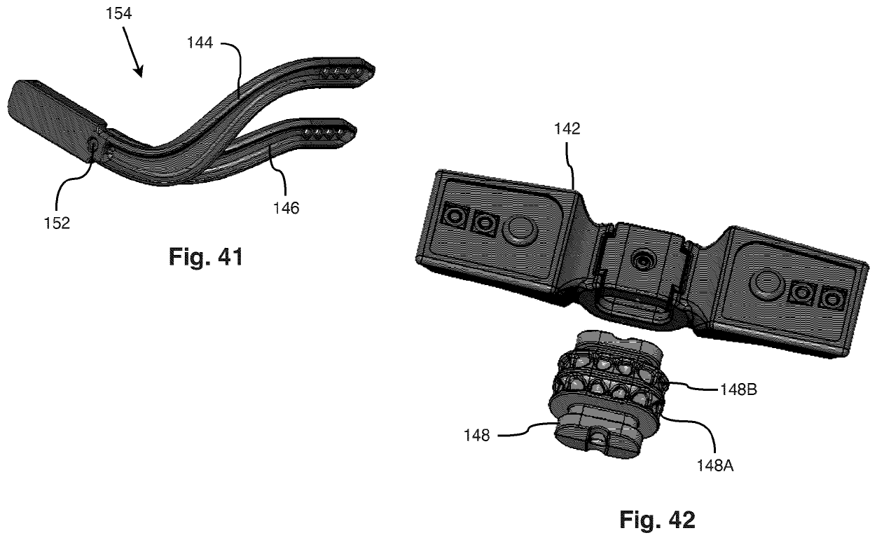

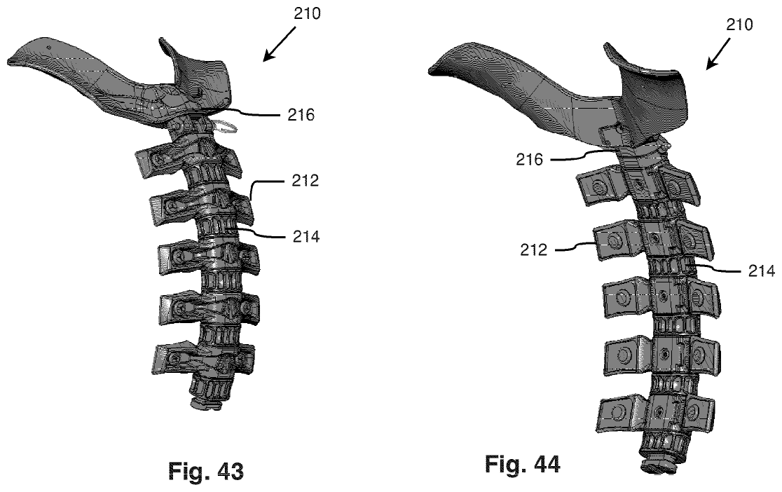

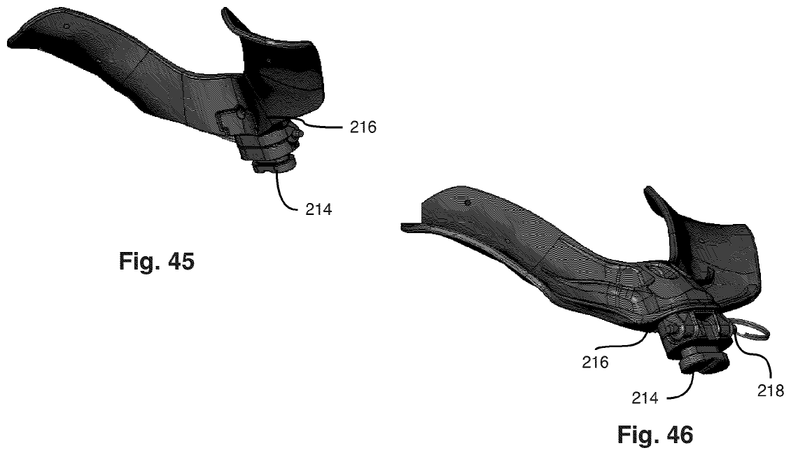

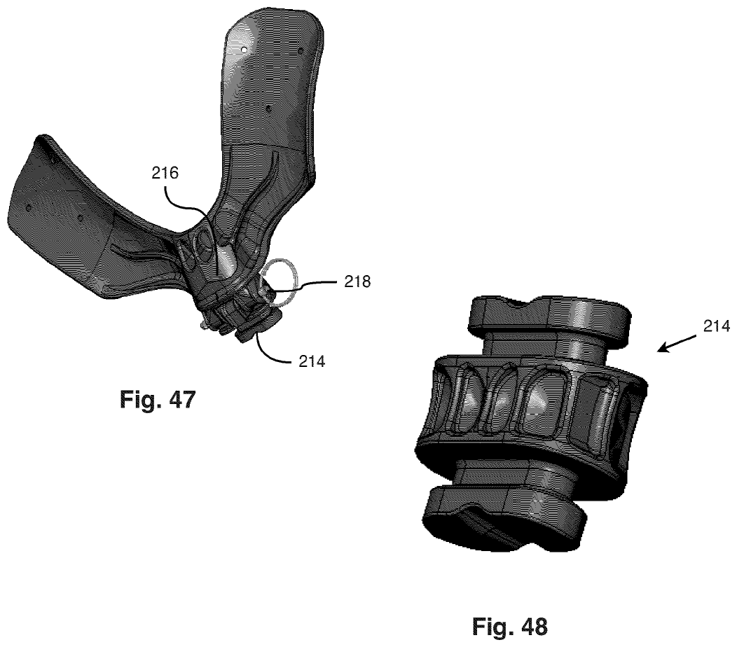

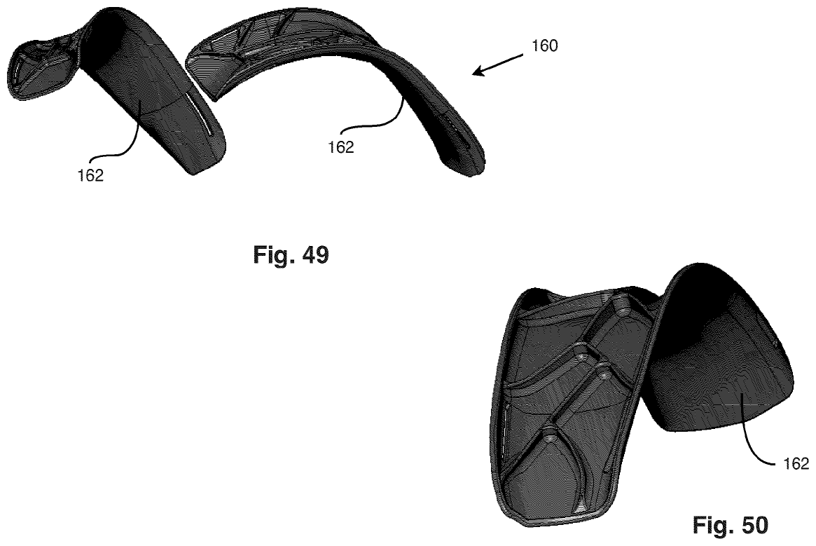

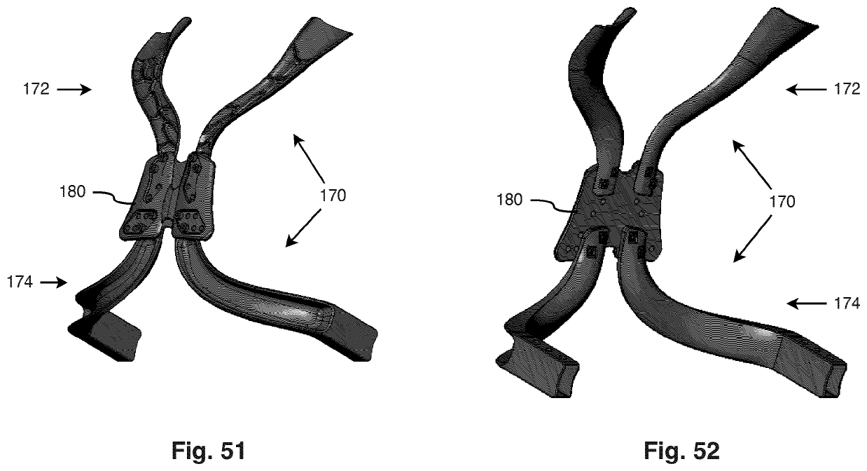

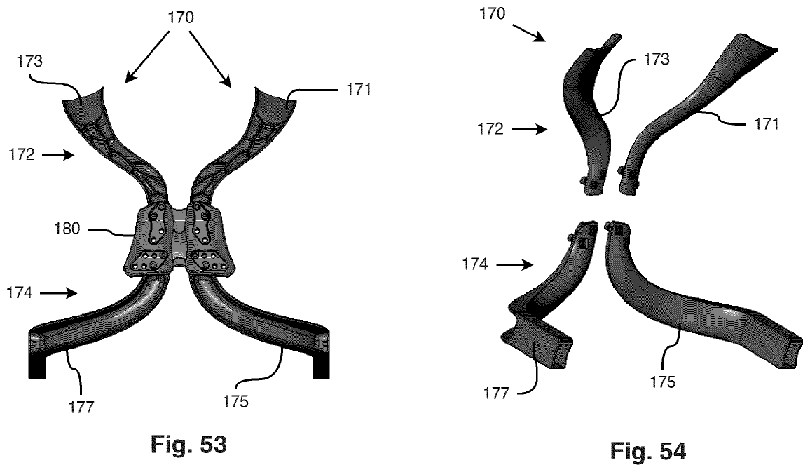

The posterior ribs component 140 comprises rib vertebrae 142 that interface with the thoracic spine component 210 and the lumbar spine component 190. In an embodiment, the rib vertebrae 142, under load, stack up one onto the other by means of a physical abutment provided by the inferior part 148A and the superior part 148B of the intervertebral joint 148. As depicted through FIGS. 39A-B and 40A-B, the rib vertebrae 142 and the intervertebral joint 148 are fixedly coupled with each other while the joint parts 148A, 148B are free to separate from each other when the posterior ribs component 140 is bent toward the anterior, and are configured to limit relative displacement through parts of the posterior ribs component 140 butting up against each other when trying to bend the posterior ribs component 140 toward the posterior and under load. To each side, rib vertebrae 142 provide an adjustable interface with a rib assembly 154 comprising an upper rib 144 and a lower rib 146. The coupling of rib vertebrae 142 is performed using the intervertebral joint 148 coupled to the rib vertebrae 142 in a similar fashion to the coupling of the lumbar intervertebral joint 194 (see e.g., FIGS. 29-30) to neighbor vertebrae. Accordingly, the rib vertebrae 142/intervertebral joint 148 interface is similar the vertebrae 192/lumbar intervertebral joints 194 interface. The posterior ribs component 140 comprises on each side an upper rib 144 and a lower rib 146 that extend laterally and toward the anterior, wrapping around the thorax of the user, from rib vertebrae 142. The upper rib 144 and the lower rib 146 are joined together by a flexion pivot 152 allowing, in cooperation with the 2-part configuration of the intervertebral joint 148, for the posterior ribs component 140 to be bent toward the anterior are to be able to resume the normal position afterwards. The upper ribs 144 prolong toward the anterior beyond the flexion pivot 152 where it is assembled to the posterior ribs component 140 by means of an adjustable interface. The posterior ribs component 140 also comprises flexion pivots 152 configured to compensate for the elongation of the surface of the spine of the user occurring with movements as well as for the structure offset from the anatomical spine of the user. The flexion pivots 152 allow a relative translation between the upper rib 144 and the lower rib 146 when the user flexes their back.

According to alternative embodiments, the posterior ribs component 140 would comprise a telescopic or a gel like component that allows for the elongation of the artificial spine of the upper body chassis 100.

Referring to FIGS. 2A-B and 43-48. the thoracic spine component 210 is a modular stack-up of vertebrae 212 and thoracic intervertebral joints 214 that allows close fitting of the user's natural kyphosis. To ensure its mechanical stability under loads. the thoracic spine component 210 is firmly connected to the shoulder bridge component 160, the anterior ribs component 170. the posterior ribs component 140, and the sternum component 180, all part of the thoracic cage 105 that defines an enclosure which ensures a closed mechanical loop. The design of the thoracic spine component 210 is inspired from the human thoracic skeleton, where the rib cage was shown to play a critical role in providing mechanical stiffness to the thoracic spine. As for other parts of the upper body chassis 100, the thoracic spine component 210 is adaptable to differences in morphology of users through intervertebral joints 214 being provided in different angulations. Furthermore, the modular stack-up configuration of the thoracic spine component 210 allows to conform to the variability of kyphosis curves of the users (different kyphosis angles and curve lengths).

The thoracic spine component 210 comprises intervertebral joints 214 that are rigid polymeric parts that are assembled to the vertebrae 212. The thoracic spine component 210 comprises vertebrae 212 that are used in the assembly of the thoracic spine component 210. The vertebrae 212 are identical to the vertebrae 192 (see e.g., FIGS. 29-30). The height and angulation of the vertebrae 212 can be adjusted to fit the user's morphology and preferences. The thoracic spine component 210 finally comprises an upper vertebra 216 that is the uppermost vertebra of the stack-up of the thoracic spine component 210. The upper vertebra 216 provides an adjustable interface with shoulder bridge component 160. The upper vertebra 216 may comprise a hinge 218 for donning and doffing purposes.

Referring to FIGS. 2A-B, 43-44 and 49-50, the shoulder bridge component 160 consists in two (2) C-shaped polymeric parts 162 that connect to the upper vertebra 216 and anterior ribs component 170 by means of hook and loop adjustment mechanisms that allow fine adaptation of the shoulder gap to the user's morphology and personal preference. One function of the shoulder bridge component 160 is to provide the main interface to the user's gear (Fragmentation Vest, LBV, Rucksack, PPE, etc.).

Referring to FIGS. 2A-B and 51-54, the anterior ribs component 170 and the sternum component 180 complete the thoracic cage 105. The anterior ribs component 170 connects to the top to the shoulder bridge component 160 by means of adjustment mechanisms as mentioned.

The anterior ribs component 170 comprises an upper-left rib 171, an upper-right rib 173, a lower-left rib 175 and a lower-right rib 177. The anterior ribs component 170 can also be divided in an upper rib portion 172 and a lower rib portion 174 coupled to each other at the center. At the center, each of the upper-left rib 171, the upper-right rib 173, the lower-left rib 175 and the lower-right rib 177 are coupled to the sternum component 180. That coupling is configured to prevent excessive deformation of the thoracic cage 105 under vertical, lateral, and antero-posterior loads.

It is worth mentioning that the sagittal and transverse geometry of the anterior ribs component 170 is carefully designed to offset the sensitive areas of the user's clavicles, sternum, and abdomen.

According to embodiments, foam paddings can be installed on the internal face of the anterior ribs component 170, where interfacing with the user's ribs and clavicles, for pressure dampening.

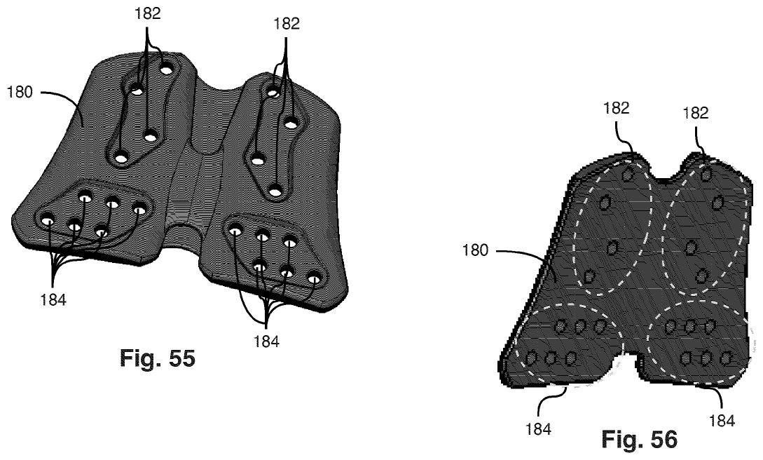

Referring additionally to FIGS. 55-56, the sternum component 180 is a central polymeric part that stabilizes the thoracic cage 105. The sternum component 180 is configured to prevent excessive deformation of the thoracic cage 105 under vertical, lateral, and antero-posterior loads. The sternum component 180 also provides adjustability in term of height and width to the thoracic cage 105 through the series of holes 182 for coupling to the upper rib portion 172 and holes 184 for coupling to the lower rib portion 174.

Through the unique configuration and characteristics of its parts, and the specific interaction of its parts. the upper body chassis 100 tends to at least minimize, and, in some embodiments, eliminate pressure points at the shoulders and chest area of the user, therethrough to reduce incidence of shoulder musculoskeletal injuries. The upper body chassis 100 allow to easily adjust local structure proximity to the skin according to user's preference.

Through its characteristic of being highly adaptable to the user's upper torso morphology, the upper body chassis 100, and particularly the adjustment hook and loop mechanisms installed in front and behind the user's shoulder, the upper body chassis 100 allows to modulate the shoulder bridge position so that the gear-to-user gap size and location are positioned precisely where the user needs it. It allows at the same time to avoid excessive gaps, and thus prevent unnecessary bulkiness while maintaining shoulder and neck mobility.

Trials demonstrated that the upper body chassis 100 is highly effective in protecting the user's shoulders from compressive forces induced by wear of the ballistic protective equipment and other load carriage or PPE. The gap created by the shoulder bridge completely unloads the user's shoulder, avoiding any pressure point or human soft tissue compression. The upper body chassis 100 also reduces compressive forces applied onto the chest (including clavicles and sternum).

Trials with the wear of a ballistic protective equipment demonstrated that the upper body chassis 100 induced reduction up to 90% of the vertical compression resulting from wearing the ballistic protective equipment and up to 43% of moments relative to the vertical axis, such reduction of moment being indicative of a decrease of pressure on the upper back of the user.

Since carriage of body armor, load bearing equipment and rucksacks was associated with shoulder and upper limbs discomfort and impairment, including weakness, pain, sensory loss, and, in some rare grave cases, in nerve damage conducting to shoulder palsy, some authors believe that “the wear of body armor and load carriage vests may increase the risk of development of rucksack palsy, despite advances in the design of the rucksack” [De Luigi. A. J., Pasquina. P., & Dahl. E. (2008). Rucksack-induced plexopathy mimicking a lateral antebrachial cutaneous neuropathy. American journal of physical medicine & rehabilitation, 87(9), 773-775]. Trials demonstrated that the upper body chassis 100 helps prevent development of shoulder injuries associated with load carriage. As mentioned, the upper body chassis 100 reduces the compression of the underlying shoulder soft tissues when the user wears the ballistic and load bearing equipment. The waist belt 110 provides a good lumbar support and allows to redirect and transfer the load carried on the torso according to the human anatomy. This important feature of the upper body chassis 100 greatly enhances the comfort of the user when wearing ballistic equipment and minimizes the occurrence of load concentration at the sacrum level.

The upper body chassis 100 delivers an unparalleled distribution of gear related loads onto the lumbo-sacral area, providing a superior comfort and support to the user.

The upper body chassis 100 distributes loads to the lower torso regions. The S-shaped spine of the upper body chassis 100, and the characteristics of it components, including the six (6) degrees of freedom of the lumbar spine component 190 deliver a revolutionary alternative approach to the notion “load transfer” provided by a worn equipment. The upper body chassis 100 selectively distributes a portion of these loads to support the lower back, and a portion of loads on the anterior aspect of sacrum. This way, the upper body chassis 100 delivers a load bearing support that functions in parallel with the natural spine-sacrum-ilia mechanics.

The upper body chassis 100 integrates a lumbar support function, thanks to the innovative structure kinetics described hereinbefore.

In practice, in unloaded neutral position, the upper body chassis 100 is adjusted so that the lumbar vertebrae 192 are in contact with the user's natural lumbar curve. When load (frag vest, LBV, etc.) are donned onto the upper body chassis 100, the Lumbar Spine component 190, in conjunction with the mobile sacrum component 120, lean slightly towards the user.

The outcome is a bracing effect provided to the user's lumbar region. Furthermore, this effect is maintained throughout the duration of tasks such as walking, as the lumbar spine component 190, through its six (6) degrees of freedom, follows the movement of the natural spine.

Similarly, through its design, the sacrum component 120 is configured to similarly adapt to changes in angle of the sacrum of the user as they e.g., walk.

The upper body chassis 100 greatly enhances thermal comfort through multiple air circulation passages integrated between gear and the user's skin.

It is recognized that convection (including evaporation), under normal high temperature exterior operational conditions, is one of the more effective pathways for heat loss in humans. Heat loss by convection requires that skin interacts with air from the external environment, which is usually cooler than the skin. When skin is covered by clothing and equipment, this air exchange can be achieved by clothing ventilation. Numerous research studies showed that clothing apertures can considerably improve clothing ventilation and therefore positively impact user's heat and moisture comfort, especially in presence of air movement (wind) or when work is performed (body movement).

When user wear multiple layers of equipment (particularly ballistic and load bearing equipment), clothing ventilation is greatly decreased, if not eliminated. Air gaps yield under the weight of gear and due to the snug fit of equipment and most of the trunk skin surface is covered by impermeable material (such as a fragmentation vest).

The upper body chassis 100 maximizes the skin surfaces that would be in contact with ambient air. When the upper body chassis 100 is worn under gear (e.g., fragmentation vest), the structure of the upper body chassis 100 slightly offsets the gear from the skin. The footprint of the upper body chassis' parts that directly contact the skin is minimized, and gap areas (in terms of skin surface) are deliberately widened.

As a result, air flow gaps were incorporated in the upper body chassis 100 on the lateral sides of the spine, at the top front and back of the shoulders, at the chest, at the abdomen, and on the sides of the torso of the user. Small apertures are generically created by the shoulder offset and chest offset.

During trials, thermal scans taken after walk sessions clearly indicated that widespread airflow gaps incorporated in the design of the upper body chassis 100 around the torso improves the airflow in these areas and results in cooler skin temperatures. Spot measurements showed a clear reduction in skin temperature on large areas. For example, a considerable skin temperature decrease was noted for the chest (decrease of 3.2 degrees Celsius) and abdomen (decrease of 5.6 degrees Celsius), and for the back torso at scapula (decrease of 4.6 degrees Celsius) and under scapula (decrease of 4.6 degrees Celsius) after a 30-minute walk wearing a fragmentation vest with an air temperature of 30 degrees Celsius and a relative humidity of approximately 42%, humidex score of 34.

The upper body chassis 100 provides a great level of modularity and adaptability to the unique morphological traits of the user as well as to the user personal preferences. Notably, the modularity of the upper body chassis 100 allows to adapt the distribution of the load being carried following the user's needs and preference. This upper body chassis 100 is further configurable to adapt to the anthropometric differences in gender, age, and ethnicity.

Mechanisms for fine adjustment discussed before are present at critical areas to minimize number of sizes and to allow day-to-day adjustments based on user's preference.

The upper body chassis 100 is configured to be able to response, and even to keep a great efficacy in response of dynamic tasks that require high user mobility and agility.

The low profile of the upper body chassis 100 is primarily designed to support Canadian Armed Forces (CAF) fragmentation protective vest (frag vest), CAF ballistic plates and CAF load bearing vest (LBV). This system could also be used to support the weight of other ballistic protection equipment, personal protective equipment, load carriage equipment, backpacks, or rucksacks. It can be adapted to other gears, and to similar gears of other military forces based on particularities of these gears. It can also be used with load carriage equipment or PPE for non-military use.

The functionalities of the upper body chassis 100 are projected to significantly improve the users' comfort and well-being, to reduce musculoskeletal injuries related to load carriage, and in turn, to increase the users' performance and well-being compared to nowadays available solutions.

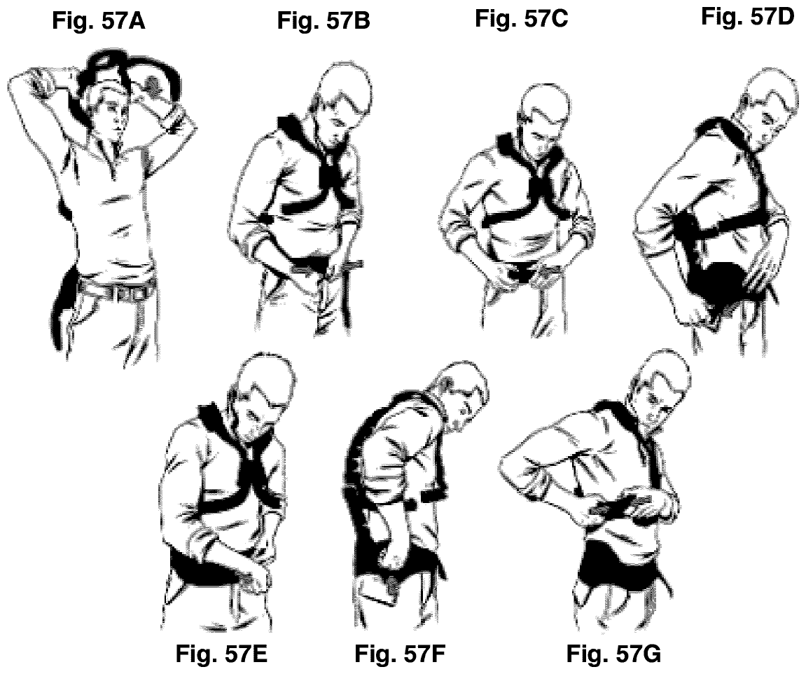

Referring to FIGS. 57A-G and 58A-D, methods of donning and doffing the upper body chassis 100

In the depicted embodiment, the UBC donning and doffing rely on 3 mechanisms: 1—front closure of the waist belt 110 (including tightness adjustment function); 2—closure of the side of the thoracic cage 105 (including tightness adjustment function); and 3—upper Intervertebral hinge.

Depicted through FIGS. 57A-G, donning steps typically comprise:

FIG. 57A: the user dons the upper body chassis 100 over their head;

FIG. 57B: the user overlaps right and left waist belt parts;

FIG. 57C: The user locks the waist belt hook (without tightening);

FIG. 57D: The user ensures the waist belt retention flaps are out of the way;

FIG. 57E: the user tightens the waist belt front closure;

FIG. 57F: the user folds back the waist belt retention flaps over the waist belt; and

FIG. 57G: the user locks and adjusts the thoracic cage side closures.

After these steps, the upper body chassis 100 is fully donned, ready to wear e.g., a fragmentation vest.

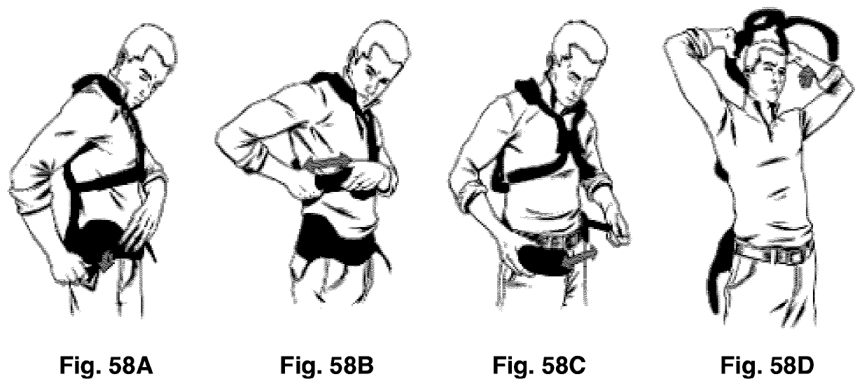

Depicted through FIGS. 58A-D, doffing steps typically comprise

FIG. 58A: the user detaches the waist belt retention flaps;

FIG. 58B: the user unlocks the thoracic cage side closures;

FIG. 58C: the user detaches the front closure; and

FIG. 58D: the user takes off the upper body chassis 100 over their head.

While preferred embodiments have been described above and illustrated in the accompanying drawings, it will be evident to those skilled in the art that modifications may be made without departing from this disclosure. Such modifications are considered as possible variants comprised in the scope of the disclosure.

1. An upper body chassis to be worn by a user, comprising:
a thoracic cage configured to at least partially enclose the user's thorax, the thoracic cage comprising shoulder bridge components and anterior ribs components extending above and below the user's shoulders to couple together up front of the user's thorax;
a pelvic cage configured to wrapped around the user's body about the user's belt line; and
a lumbar spine component coupling the pelvic cage to the thoracic cage, the lumbar spine component comprising:
a plurality of vertebrae; and
at least one intervertebral joint configured to couple two of the plurality of vertebrae, the intervertebral joint comprising a body providing a plurality of degrees of freedom for movement of the coupled vertebrae relative to each other.

2. The upper body chassis of claim 1, wherein the plurality of vertebra comprises at least three vertebra, and the at least one intervertebral joint comprises at least two intervertebral joints.

3. The upper body chassis of claim 1, wherein the thoracic cage comprises a first spine section configured to extend in a caudal-cranial direction along the user's spine, the first spine section being configured to be incompressible in the caudal-cranial direction.

4. The upper body chassis of claim 3, wherein the thoracic cage comprises a first side ribs assembly comprising:
an upper rib and a lower rib extending laterally and toward an area anterior to the first spine section, the upper rib and the lower rib being rigidly coupled to the first spine section, and
a rib coupling configured to couple the upper rib to the lower rib while providing at least one degree of freedom.

5. The upper body chassis of claim 4, wherein the rib coupling is a pivotable coupling.

6. The upper body chassis of claim 4, comprising a second side ribs assembly, wherein the first side ribs assembly and the second side ribs assembly extend over opposite sides of the user, and wherein the first side ribs assembly and the second side ribs assembly are coupled to each other at the user's front, thereby wrapping around the user's thorax.

7. The upper body chassis of claim 1, wherein the lumbar spine component comprises a second spine section component configured to extend in a caudal-cranial direction along the user's spine, the second spine section being configured to be:
a) at least one of i) expandable and ii) compressible in the caudal-cranial direction; and
b) rotatable around a connection line connecting one of the vertebrae to one of the intervertebral joint, the connection line extending in the caudal-cranial direction.

8. (canceled)

9. The upper body chassis of claim 1, wherein the intervertebral joint is configured to prevent the lumbar spine component to bend toward a posterior area while allowing the lumbar spine component to bend toward an anterior area through caudal-cranial move of the vertebrae away from each other.

10. The upper body chassis of claim 1, wherein the pelvic cage comprises a sacrum component coupled to the lumbar spine component, the sacrum component being configured to butts up against the user's sacrum and to be able to adapt to position and angle of the user's sacrum as the user moves.

11. The upper body chassis of claim 10, wherein the pelvic cage comprises a waist wrapping component, coupled to the sacrum component, configured to wrap around the user's waist thereby maintain the sacrum component abutting the user's sacrum.

12. The upper body chassis of claim 11, wherein the waist wrapping component comprises—two belt extremities, a closure component configured to releasably couple and maintain coupling of the two belt extremities, and a 3D shape assembly of strands configured for the waist belt to interface with a plurality of:
a depression of the user's sacral region;
protrusions of the user's iliac crests;
the user's anterior inferior iliac spines (ASIS); and
a depression under the user's abdomen.

13. (canceled)

14. (canceled)

15. The upper body chassis of claim 10, wherein the sacrum component comprises:
a sacrum lower body configured to interface with the user's sacrum;
a sacrum upper body rotatably coupled to the sacrum lower body; and
a coupling component mounted to the sacrum upper body, configured to receive a load from the lumbar spine component.

16. The upper body chassis of claim 10, wherein the sacrum component comprises a sacrum body and a pair of wings extending laterally from the sacrum body, the wings being coupled pivotably to the sacrum body, wherein the wings are configured to interface with the user's back.

17. The upper body chassis of claim 16, wherein the sacrum body comprises a sacrum lower body and a sacrum upper body coupled rotatably to each other;
wherein rotatable coupling of the sacrum upper body and sacrum lower body defines a first axis;
wherein rotatable coupling of the wings to the sacrum body defines a second axis; and
wherein the first axis and the second axis are parallel.

18. The upper body chassis of claim 1, wherein the intervertebral joint comprises:
a first extremity and a second extremity; and
a body connecting the first extremity to the second extremity, the body providing a plurality of degrees of freedom to the intervertebral joint for movement of the extremities relative to each other.

19. The upper body chassis of claim 1, wherein the vertebrae comprises a first element and a second element configured to be assembled together to couple the vertebrae to the intervertebral joint.

20. The upper body chassis of claim 1, wherein the plurality of vertebrae are configured to adjust so that when a load is donned onto the upper body chassis, the lumbar spine component leans slightly towards the user's spine.

21. The upper body chassis of claim 1, wherein the thoracic cage comprises a thoracic spine component, the shoulder bridge components, the anterior ribs components comprising a left-side ribs assembly and a right-side ribs assembly, and a sternum component;
wherein the shoulder bridge components are connected to a first portion of the thoracic spine component, and are configured to wrap the use's thorax above the user's shoulders;
wherein the left-side ribs assembly and the right-side ribs assembly are connected to a second portion of the thoracic spine component that is lower than the first portion, the left-side ribs assembly and the right-side ribs assembly being configured to wrap around the user's thorax about the user's ribs; and
wherein the shoulder bridge components, the left-side ribs assembly, the right-side ribs assembly couple to the sternum component about the user's sternum.

22. An upper body chassis to be worn by a user, comprising:
a thoracic cage configured to at least partially enclose the user's thorax;
a pelvic cage configured to wrapped around the user's body about the user's belt line; and
a lumbar spine component coupling the pelvic cage to the thoracic cage, the lumbar spine component comprising:
a plurality of vertebrae; and
at least one intervertebral joint configured to couple two of the plurality of vertebrae, the intervertebral joint comprising a body providing a plurality of degrees of freedom for movement of the coupled vertebrae relative to each other,
wherein the intervertebral joint is configured to prevent the lumbar spine component to bend toward a posterior area while allowing the lumbar spine component to bend toward an anterior area through caudal-cranial move of the vertebrae away from each other.

23. An upper body chassis to be worn by a user, comprising:
a thoracic cage configured to at least partially enclose the user's thorax;
a pelvic cage configured to wrapped around the user's body about the user's belt line; and
a lumbar spine component coupling the pelvic cage to the thoracic cage, the lumbar spine component comprising:
a plurality of vertebrae; and
at least one intervertebral joint configured to couple two of the plurality of vertebrae, the intervertebral joint comprising a body providing a plurality of degrees of freedom for movement of the coupled vertebrae relative to each other,
wherein the pelvic cage comprises a sacrum component coupled to the lumbar spine component, the sacrum component being configured to butts up against the user's sacrum and to be able to adapt to position and angle of the user's sacrum as the user moves, and
wherein the sacrum component comprises a sacrum body and a pair of wings extending laterally from the sacrum body, the wings being coupled pivotably to the sacrum body, wherein the wings are configured to interface with the user's back.
